(19) United States
(12) Patent Application Publication
(10) Pub. No.: US 2012/0156766 A1
(43) Pub. Date: Jun. 21, 2012

(54) SAMPLE ANALYZING CHIP AND MEASUREMENT SYSTEM USING SAME

(76) Inventors: Akiko Shiratori, Tokyo (JP); Yoshiaki Yazawa, Nishitokyo (JP); Takeshi Fujita, Niiza (JP); Kunio Harada, Hachioji (JP); Kenko Uchida, Tokyo (JP)

(21) Appl. No.: 13/379,696

(22) PCT Filed: Jun. 15, 2010
(86) PCT No.: PCT/JP2010/060094

§ 371 Date: Dec. 21, 2011

(30) Foreign Application Priority Data
Jun. 24, 2009 (JP) 2009-149294

(51) Int. Cl. C12M 1/40; G01N 21/76

(52) U.S. Cl. 435/287.2; 422/70

(57) ABSTRACT

Temperature of a sensor chip itself rises with the supply of power owing to temperature dependence of the sensor chip as its basic characteristics. When a chemiluminescence reagent is added at the point of time at which the temperature rise reaches a steady state and a sensor chip photodiode dark current becomes constant, a drastic shift occurs in the sensor chip temperature. Remarkable dispersion occurs at this time in the sensor chip photodiode dark current Variance (unstability) of the sensor chip photodiode dark current can be decreased by reducing the temperature fluctuation of the sensor chip to minimum by using an exothermal effect of a thermal diffusion medium.

This application is based upon and claims priority of Japanese Patent Application No. 2009-149294, filed Jun. 24, 2009, the content of which is incorporated therein by reference.

### TECHNICAL FIELD

This invention relates to a small device for detecting or measuring an immunological reaction and a chemical reaction by using a sensor chip having a wireless communication function and a photo sensor function mounted thereto.

### BACKGROUND ART

Devices capable of high sensitivity quantitative assay by utilizing a color reaction and an agglutination reaction for a detection system of an immunological reaction and a chemical reaction and an optical system including a light source (LED: Light Emitting Diode) and a sensor (PD: Photo Diode) for detection are known as prior art technologies. Panel assay devices using chemiluminescence for the detection system have also been merchandized.

Patent Literature 1 discloses a measuring device that fixes a probe for a biological substance onto a chip on which a sensor and a functional block having a wireless transmission/reception function are formed, detects a supplemented target by the sensor and transmits the sensing result by the wireless transmission/reception function to an external controller.

### CITATION LIST

### Patent Literature

Patent Literature 1: JP-A-2004-0101253

### SUMMARY OF INVENTION

### Technical Problem

Intensive test apparatuses installed in large-scale hospitals and test centers have been utilized for the laboratory tests of various kinds of proteins, virus and bacteria as markers of disorders to reduce cost by labor saving. On the other hand, POCT (Point of Care Testing) has been spread steadily in emergency tests such as emergent outpatient tests and tests in intensive care units for its rapidness to provide the test results on site and easiness of tests, tests for infectious diseases for outpatients inspection and self tests (e.g. blood sugar value test at home) for which convenience and compactness are requisite. High sensitivity has also been required for POCT in addition to rapidness, convenience and compactness with the expansion of its utilization. To satisfy this requirement, POCT devices combining a signal detection system of sensors using a semiconductor integrated circuit technology with MEMS (Micro Electric Mechanical System) with a reaction system for detection (antigen-antibody reaction, enzyme reaction, nucleic acid hybridization reaction) have been proposed.

The problem that hereby occurs is temperature fluctuation in the POCT devices. High sensitivity of the POCT device can be achieved by applying an integration sensor chip fabricated by the semiconductor integrated circuit technology and the MEMS technology described above to the POCT device (Patent Literature 1, for example). However, the temperature of the integration sensor rises owing to electromagnetic energy supplied to operate the integration sensor chip and the temperature fluctuation of the POCT device including a reagent solution and a sample solution is unavoidable Besides the problem of exothermy in the integration sensor chip fabricated by integrating amplifiers and control circuits by the integrated circuit technology and the MEMS technology in addition to the sensor element as a single device, addition of a solution and reaction heat are also the factors of temperature fluctuation in the POCT device in which the sensor element itself is free from exothermy, too, when a reaction unit for executing various kinds of chemical and biological reactions for detecting an object substance is integrated with the sensor. Such temperature fluctuation exerts influences on the sensor element, the amplifier, the control circuit or the chemical/biological reactions and results in deterioration of sensitometry and measuring accuracy.

Exothermy of the integration sensor chip results from Joule heat and let's consider temperature fluctuation resulting from Joule heat. Means for supplying electromagnetic energy to the POCT device may either wire means or wireless means but an example of the wireless supply will be hereby picked up. The device of Patent Literature 1 supplies electromagnetic energy by wireless means from an external reader to the integration sensor chip. The sensor chip temperature rises with the supply of power by inductive coupling between the reader side coil and the integration sensor side coil. The temperature of the sensor chip itself readily changes as the sensor chip receives influences of an external environment and external factors besides the characteristics described above. FIG. 9 shows an examination result of the temperature shift of the sensor chip in sensor-chip immunochromatography.

Horizontal axis in FIG. 9 represents the time and vertical axis represents the temperature shift measured by use of a sensor chip having a thermo sensor mounted thereto. The room temperature is 26° C. at the start of the supply of electric power and 12 minutes later at which the temperature of the sensor chip itself becomes substantially constant (steady state; T1), the sensor chip temperature rises up to 57.6° C. (mean value of three measurements). When a chemiluminescent substrate solution for starting the chemiluminescence reaction is added at this point (T2), the temperature of the sensor chip itself drastically drops down to 343.6 C (mean value of three measurements) depending on the temperature of the solution added. The temperature (T3) during the subsequent chemiluminescence reaction (mean of 800 to 1,600 seconds from the start of measurement) is 40.8° C. (mean value of three measurements). Though the chemiluminescence measurement is started simultaneously with the addition of the chemiluminesnt substrate solution, a dark current of a sensor-chip photo diode fluctuates owing to drastic temperature fluctuation of the sensor chip occurring at this time and this causes measurement dispersion. The temperature fluctuation results also in unstability of the chemical/biological reaction.

The technical problem that the invention is to solve is the problem that the sensor chip photo diode dark current becomes unstable and measurement variance increases when the temperature rises due to exothermy of the sensor chip itself and the occurrence of temperature fluctuation due to external factors.

### Solution to Problem

To solve the problem described above, the invention diffuses sensor chip heat by utilizing an exothermal operation of a thermal diffusion medium to decrease temperature fluctuation and stabilizes the sensor characteristics and the chemical-biological reaction. More concretely, a heat conduction material is brought into thermal contact with the sensor chip and a sample hold-back carrier, and heat generated from the sensor chip is diffused through the heat conduction material to suppress the temperature rise of the sensor chip itself and temperature fluctuation. In other words, a sample assay chip and a system have the following features.

(1) A sample assay chip comprising a hold-back carrier for holding an immobilized sample; a sensor for detecting the reaction between a sample of a measuring object and the immobilized sample; and a thermal diffusion medium for diffusing heat generated from the sensor; wherein the thermal diffusion medium keeps thermal contact with the sensor.
(2) A sensing system comprising a sample assay chip including a hold-back carrier for holding an immobilized sample, a sensor for detecting a reaction between a sample of a measuring object and the immobilized sample and a thermal diffusion medium for diffusing heat generated from the sensor, keeping thermal contact with the sensor; and an external controller for exchanging signals with the sample assay chip.

### Advantageous Effects of Invention

The invention can suppress the temperature rise and temperature fluctuation by diffusing heat occurring from the sensor chip itself. The invention provides the effect that the sensor characteristics can be stabilized by suppressing the temperature rise of the sensor chip and temperature fluctuation. The second effect is that photo diode sensitivity can be improved as the temperature rise of the sensor chip itself is suppressed. The third effect is that because exothermy of the sensor chip is suppressed, the temperature rise of reactions (protein-protein interaction, nucleic acid hybridization, enzyme reaction, etc) occurring on the biological sample hold-back carrier keeping close contact with the sensor chip can be suppressed and the reactions can be stabilized and optimized.

Other objects, features and advantages of the invention will become more apparent from the following description of embodiments thereof taken in conjunction with the accompanying drawings.

### BRIEF DESCRIPTION OF DRAWINGS

FIG. 1 is a view showing an exothermal structure when a thermal diffusion medium is applied.

FIG. 2A is a view showing a structure when a sensor chip having a signal circuit in integration is applied to an exothermal structure.

FIG. 2B is a view showing similarly a structure when a sensor chip having a signal circuit in integration is applied to an exothermal structure.

FIG. 3A is a view showing a structure when a sensor chip having a signal circuit and a wireless communication circuit that are integrated is applied to an exothermal structure.

FIG. 3B is a view showing similarly a structure when a sensor chip having a signal circuit and a wireless communication circuit that are integrated is applied to an exothermal structure.

FIG. 3C is a view showing similarly a structure when a sensor chip having a signal circuit and a wireless communication circuit that are integrated is applied to an exothermal structure.

FIG. 4A is a view showing an example of the arrangement of a thermal diffusion medium.

FIG. 4B is a view similarly showing an example of the arrangement of a thermal diffusion medium.

FIG. 5 is a view showing a structure when a biological sample hold-back carrier is introduced to an exothermal structure.

FIG. 6 is a view showing a structure when a plurality of sensor chips is applied to an exothermal structure.

FIG. 7 is a view showing a structure when a thermal diffusion medium and a sensor chip are integrated.

FIG. 8 is a view showing a structure when a biological sample hold-back carrier and a sensor chip are integrated.

FIG. 9 is a view showing an exothermal effect of a thermal diffusion medium.

FIG. 10A is a view showing a structure in sensor-chip immunochromatography.

FIG. 10B is a view showing similarly a structure in sensor-chip immunochromatography.

FIG. 11A is a view showing an exothermal structure in sensor-chip immunochromatography.

FIG. 11B is a view showing similarly an exothermal structure in sensor-chip immunochromatography.

FIG. 12A is a view showing a diffusion effect (temperature shift of sensor chip) of sensor chip heat by a thermal diffusion medium.

FIG. 12B is a view showing similarly a diffusion effect (temperature shift of sensor chip) of sensor chip heat by a thermal diffusion medium.

FIG. 12C is a view showing similarly a diffusion effect (temperature shift of sensor chip) of sensor chip heat by a thermal diffusion medium.

FIG. 12D is a view showing similarly a diffusion effect (temperature shift of sensor chip) of sensor chip heat by a thermal diffusion medium.

FIG. 12E is a view showing similarly a diffusion effect (temperature shift of sensor chip) of sensor chip heat by a thermal diffusion medium.

FIG. 13 is a view showing a comparison result of signal intensity in sensor-chip immunochromatography when a heat conduction sheet is applied.

FIG. 14 is a view showing a comparison result of measurement accuracy in sensor-chip immunochromatography when a heat conductive sheet is applied.

FIG. 15 is a view showing a comparison result of sensor output dispersion of a test area in sensor-chip immunochromatography when a heat conductive sheet is applied.

FIG. 16 is a view showing a comparison result of sensor output dispersion of a blank area (photodiode dark current) in sensor-chip immunochromatography when a heat conductive sheet is applied.

FIG. 17A is a view showing a sensor-chip immunochromatography sensing structure using a resin base material.

FIG. 17B is a view showing similarly a sensor-chip immunochromatography sensing structure using a resin base material.

FIG. 18A is a view showing a temperature shift of a sensor chip in sensor-chip immunochromatography using a resin base material.

FIG. 18B is a view showing similarly a temperature shift of a sensor chip in sensor-chip immunochromatography using a resin base material.

FIG. 19A is a view showing a result in sensor-chip immunochromatography using a resin base material.

FIG. 19B is a view showing similarly a result in sensor-chip immunochromatography using a resin base material.

FIG. 20A is a view showing improvement effects of enhanced diffusion of a chemiluminescence substrate solution and its uniformity by a wrap film.

FIG. 20B is a view showing similarly improvement effects of enhanced diffusion of a chemiluminescence substrate solution and its uniformity by a wrap film.

FIG. 20C is a view showing similarly improvement effects of enhanced diffusion of a chemiluminescence substrate solution and its uniformity by a wrap film.

FIG. 21 is a view showing a structure when a high-magnetic permeable material is applied to an exothermal structure.

FIG. 22A is a view showing a structure in sensor-chip immunochromatography using a resin base material.

FIG. 22B is a view showing similarly a structure in sensor-chip immunochromatography using a resin base material.

### DESCRIPTION OF EMBODIMENTS

FIG. 1 has a feature in that a sensor chip 101, a biological sample hold-back carrier 102 and thermal diffusion medium 103 keep thermal contact. To further improve the thermal diffusion effect, these members preferably keep close contact. Reference numeral 104 denotes an antibody immobilized area. This FIG. 1 shows that the sensor chip 101 keeps a close contact not only with the thermal diffusion medium 103 for diffusing the heat generated by itself but also with the biological sample hold-back carrier 102 that serves as the reaction area of the biological sample as a measurement object. An antibody, for example, is fixed as the immobilized sample to the biological sample hold-back carrier 102 for the biological sample as the measurement object. The antibody immobilized area 104 on the biological sample hold-back carrier 102 and the sensor chip 101 are arranged in such a manner as to keep close contact with each other and the thermal diffusion medium 103 is brought into close contact with the sensor chip 101. Accordingly, this is the structure that brings the biological sample hold-back carrier 102, the sensor chip 101 and the thermal diffusion medium 103 into thermal contact with one another and to diffuse the heat generated by the sensor chip through the thermal diffusion medium.

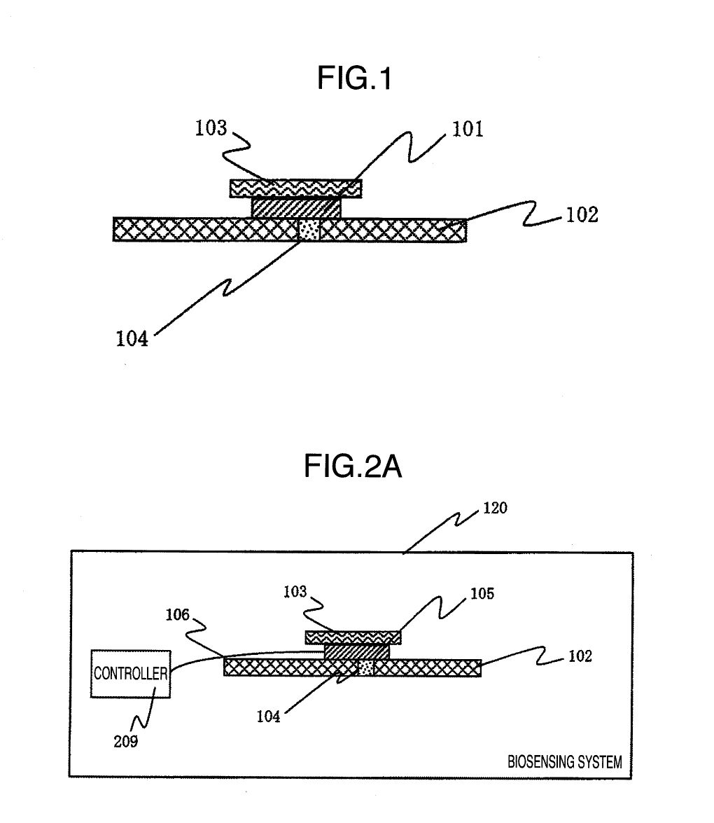

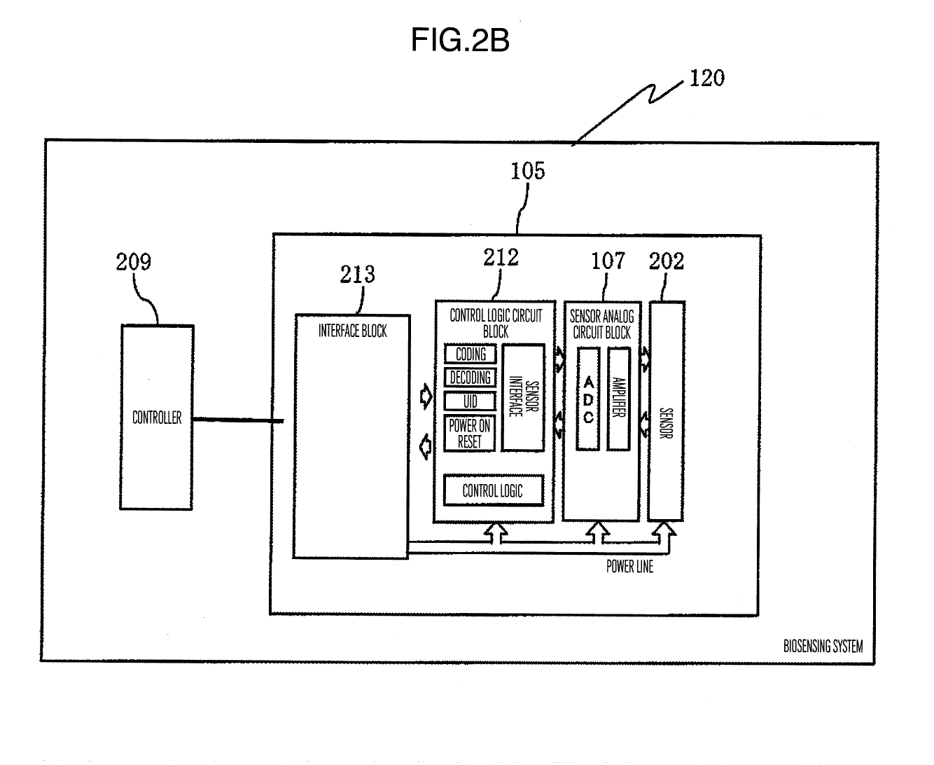

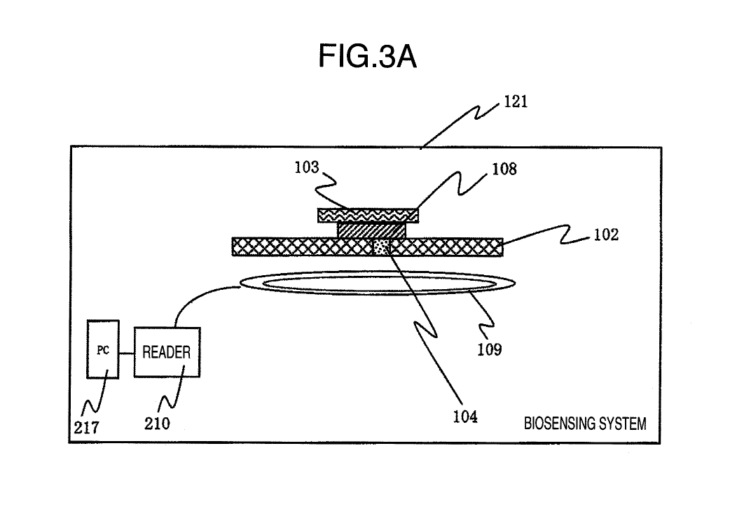

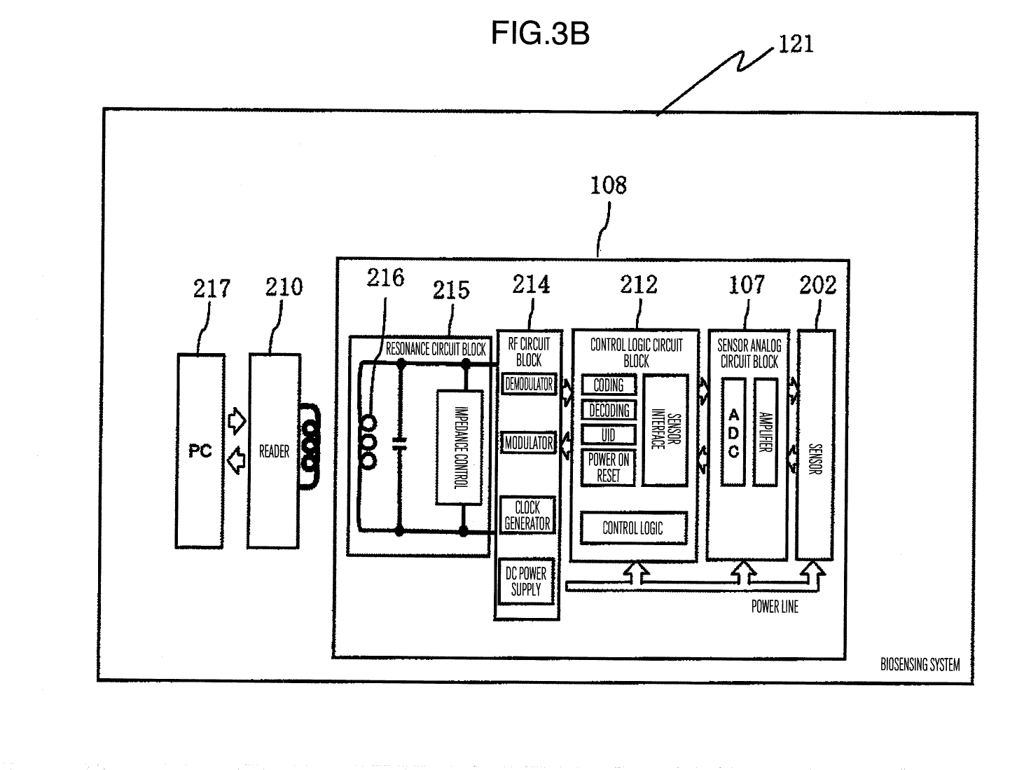

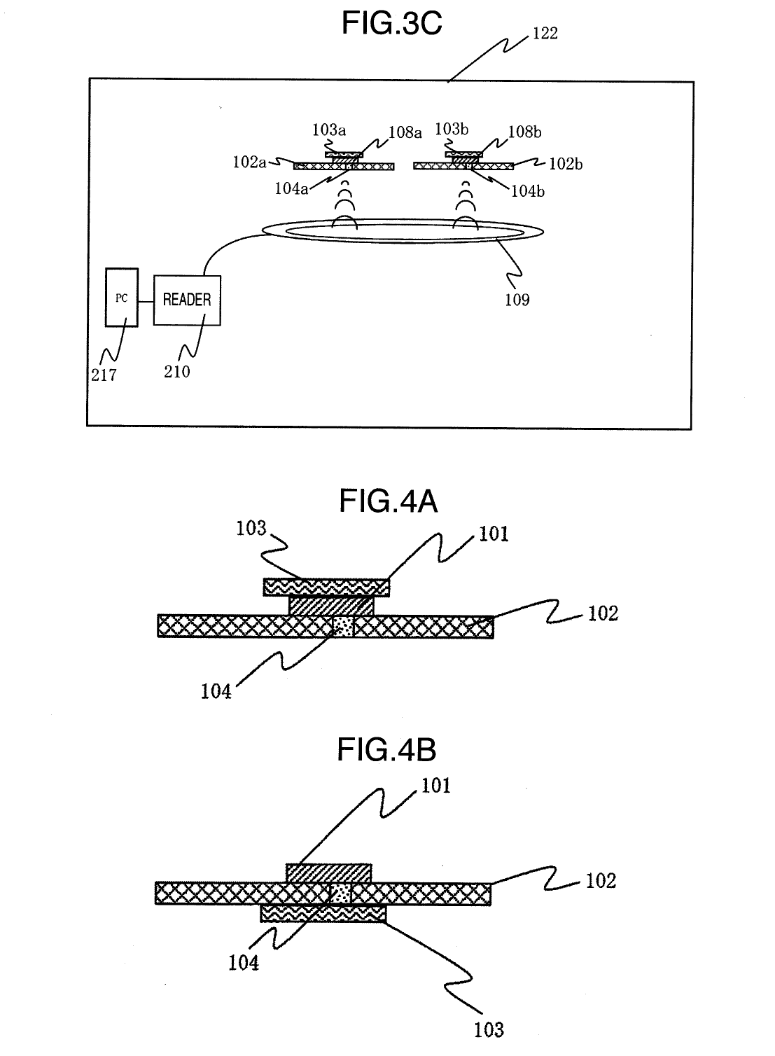

A sensor chip 105 constituted by integrating a sensor area 202, a sensor analog circuit 107 for signal processing, a control logic circuit 212 and an interface circuit 213 as shown in FIGS. 2A and 2B or a sensor chip 108 constituted by integrating an RF circuit 214 and a resonance circuit 215 in addition to the sensor area 202, the sensor analog circuit 107 for signal processing, the control logic circuit 212 and the interface circuit 213 as shown in FIG. 3 may be used, too.

The thermal diffusion medium 103 is preferably brought into close contact with the sensor chip 101 as shown in FIG. 4A. However, when the thermal diffusion medium 103 is brought into close contact with the biological sample hold-back carrier 102 as shown in FIG. 4B, exothermy of the sensor chip 101 can be diffused through the biological sample hold-back carrier 102.

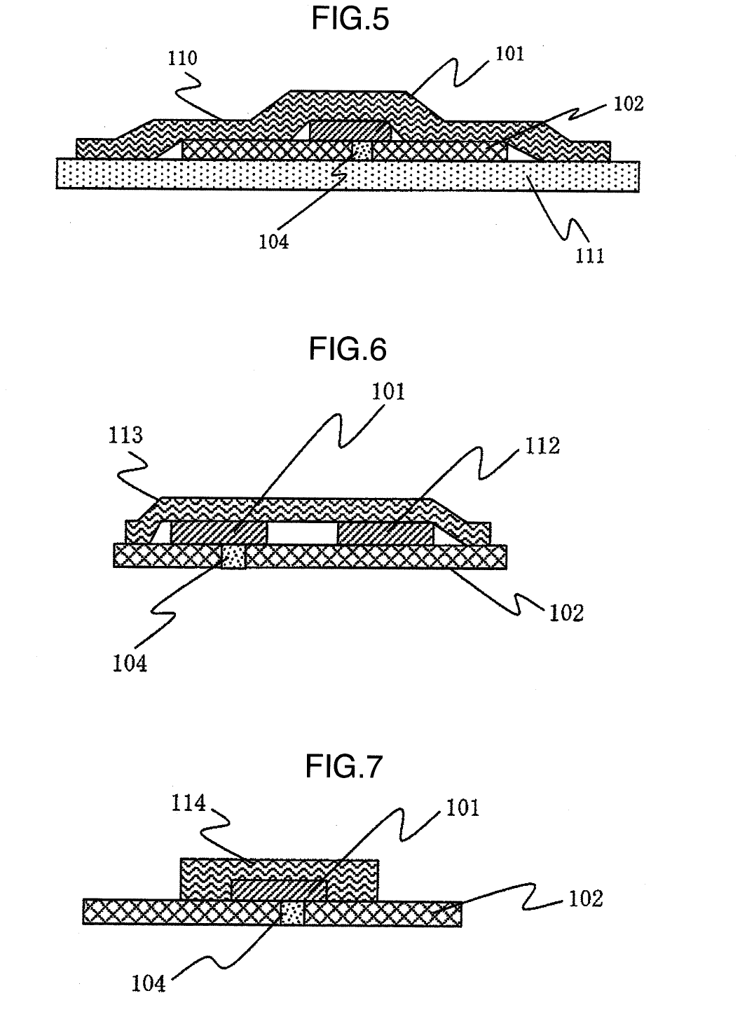

Sensor chip heat can be diffused efficiently from a supporting base 111 by bringing the thermal diffusion media 110 into close contact with the sensor chip 101, the biological sample hold-back carrier 102 and the supportive base 111 as shown in FIG. 5.

When a plurality of sensor chips is used, the thermal diffusion medium 113 is uniformly brought into thermal contact with these sensor chips to uniformly diffuse heat generated from the respective sensor chips 101 and 112 as shown in FIG. 6

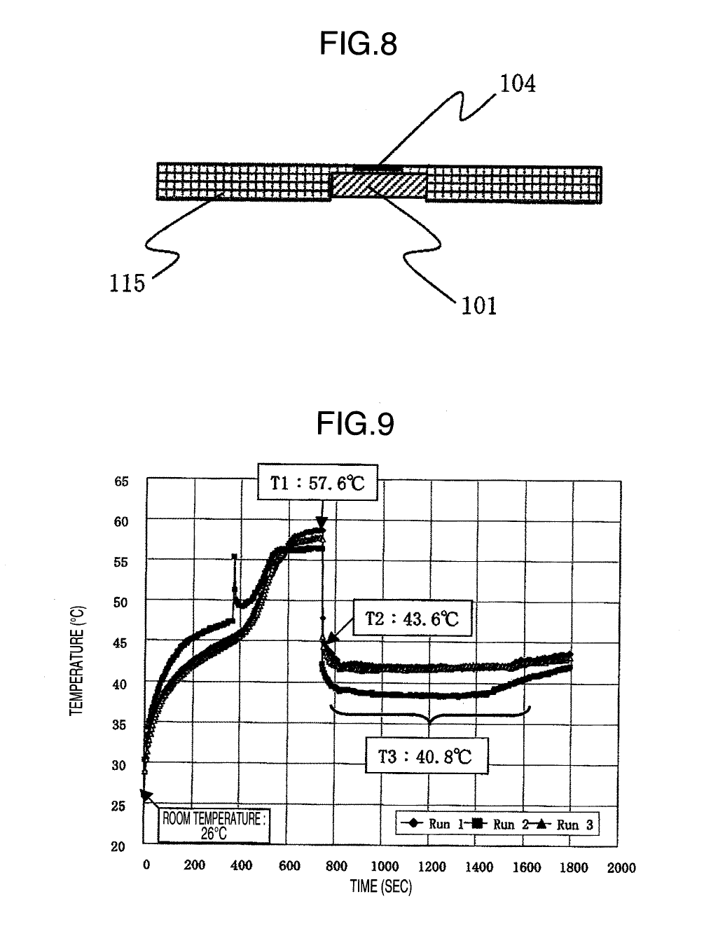

Improvement of exothermal efficiency and uniformity of heat can be achieved by employing the structure in which the sensor chip 101 keeps not only the physical contact with the thermal diffusion medium 114 but is also integrated by assembling the sensor chip 101 into the thermal diffusion medium 114 as shown in FIG. 7 or the structure in which the thermal diffusion medium 114 and the biological sample hold-back carrier 102 are integrated to provide an exothermic type biological sample hold-back carrier 115 as shown in FIG. 8. Here, an immobilized antibody is immobilized to a part 104 of the exothermic type biological sample hold-back carrier.

### Embodiment 1

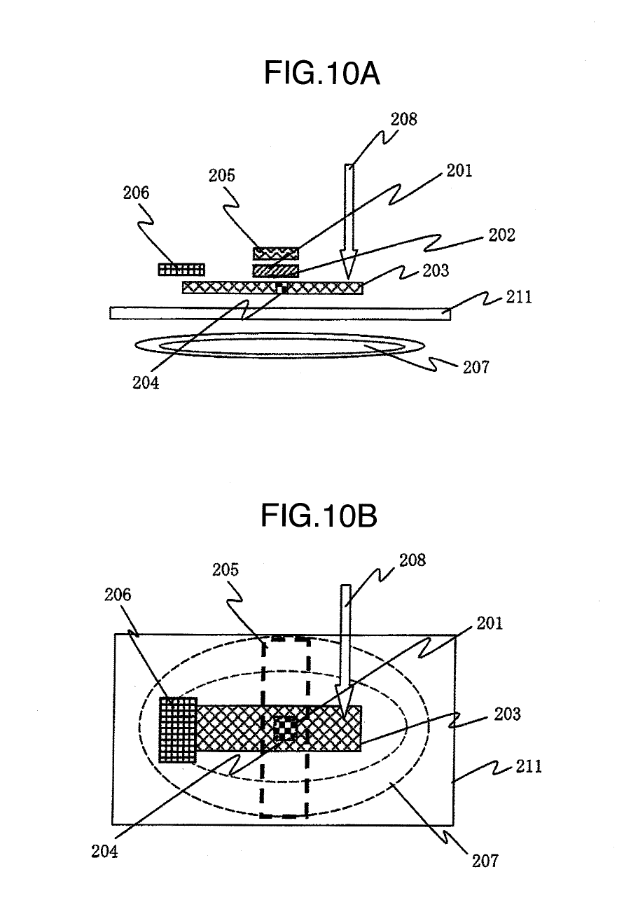

To begin with, a basic exothermal structure of immunochromatography applying a sensor chip will be explained with reference to FIGS. 10A and 10B. FIG. 10A is a sectional view of an exothermal structure and FIG. 10B is a plan view. An antibody to a biological sample as a measurement object is immobilized to a biological sample hold-back carrier 203. A sensor chip 201 is arranged in such a manner that an antibody immobilized area 204 on the biological sample hold-back carrier 203 is in conformity with a sensor chip area 202. A thermal diffusion medium 205 is brought into close contact with the sensor chip 201 (with a small space secured in the drawing for ease of understanding). The thermal diffusion sample hold-back carrier 205 keeps close contact not only with the sensor chip 201 and the biological sample hold-back carrier 203 but also with a biological sample supportive base 211. Accordingly, the biological sample hold-back carrier 203, the sensor chip 201 and the thermal diffusion medium 205 are brought into thermal contact and heat generated from the sensor chip 201 is diffused by the thermal diffusion medium. A biological sample and a reagent that are charged from an inlet 208 diffuse inside the biological sample hold-back carrier 203 and are absorbed by an absorbent pad 206. Consequently, the biological sample and the reagent react with the antibody on the antibody immobilized area 204 at a velocity that is dependent on the characteristics of the biological sample hold-back carrier 203. Further, the non-reacted reagent and biological sample are quickly absorbed by the absorbent pad 206.

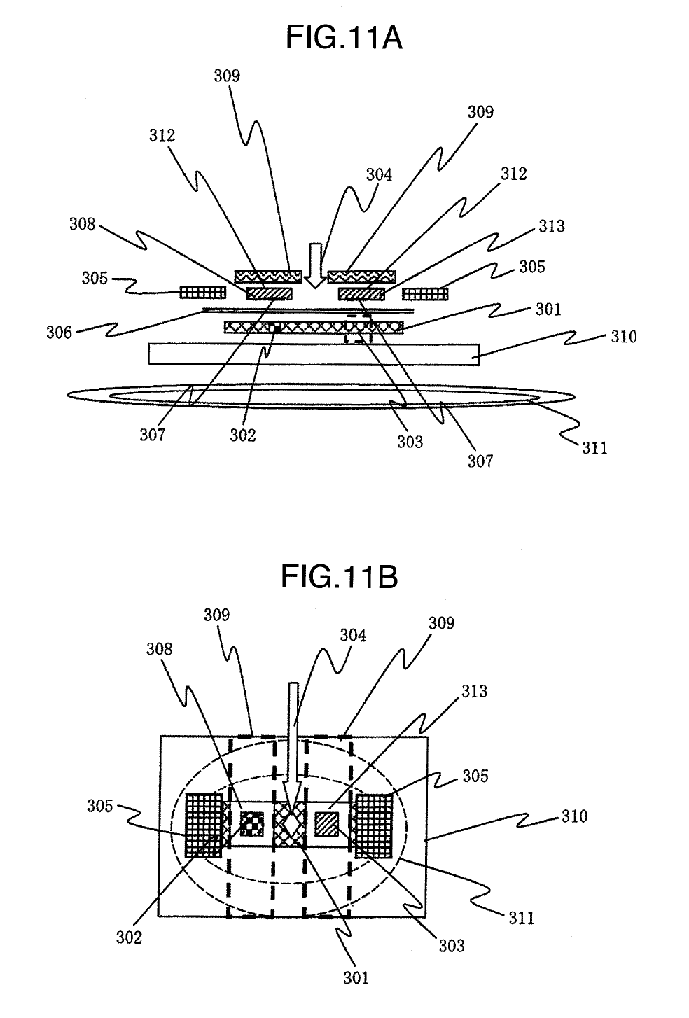

Next, Embodiment 1 will be explained by referring to the immunochromatography exothermal structure applying the sensor chip shown in FIG. 11.

The present invention executes immunochromatography that is directed to human chorionic gonadotorin: hCG) as a measurement object and applies a sandwich method utilizing Human Chorionic Gonadtropin Anti-alpha subunit 6601 SPR-5, Medix Biochemica as an immobilized antibody and Human Chorionic Gonaftropin Monoclonal Anti hcg-13, 5008SP-5, Medix Biochemica as an alkaline phosphatase modified antibody. Chemiluminescence using 1,2-Dioxetane system chemiluminescent substrate (CDP-Star, Tropix) is applied as a sensing method.

A test area 302 of a membrane component 301 applied as the biological sample hold-back carrier is the area to which the antibody to the biological sample as the measurement object is immobilized and a blank area 303 is the area to which the antibody to the biological sample as the measurement object is not immobilized. The blank area 303 is based on the premise that the diffusion condition from a solution inlet 304 and the chemiluminescence condition are the same as those at the test area 302. In FIGS. 11A and 11B, the area positioned symmetrically to the test area 302 while interposing the solution inlet 304 between them is the blank area 303.

In FIGS. 11A and 11B, a membrane component 301 using PES (polythersulfone) and having a pore diameter of 0.8 μm is applied as the biological sample hold-back carrier. A center area of the membrane component is used as the solution addition area 304 and absorbent pads 305 are arranged respectively at both ends of the membrane component 301 to absorb an excessive solution oozing out from the membrane component 301. A wrap film 306 (material: polypropylene/nylon, thermal conductivity: polypropylene 0.17˜0.19 W/m·K, nylon 0.24 W/m·k) is arranged on the membrane component 301 so as to enhance diffusion of the biological sample and the reagent and to prevent drying of the biological sample hold-back carrier. A test sensor chip 308 is arranged from above the wrap film 306 in such a manner that the sensor area 307 comes into close contact on the test area 302. Similarly, a blank sensor chip 13 is arranged in such a manner as that sensor area 307 comes into close contact on the blank area 303.

In FIG. 11, the sensor chip having a photo-sensor mounted thereto and a wireless communication function is applied so as to detect the chemiluminescence reaction resulting from alkaline phosphotase. A heat conduction sheet (material: silicone gel, thermal conductivity: 0.8 W/m·k) having a rectangular shape (25×2.5 mm) is arranged on the sensor chips 308 and 313 and a part of the heat conduction sheet 309 is brought into close contact with a base 310 (material: glass, thermal conductivity: 1.0 W/m·k) supporting the membrane component 301. The exothermal structure of the sensor chip heat shown in FIGS. 11A and 11B is the structure having a feature in that three members, that is, the membrane component 301 as the biological sample hold-back carrier, the sensor chips 308 and 313 and the heat conduction sheet, keep thermal contact with one another.

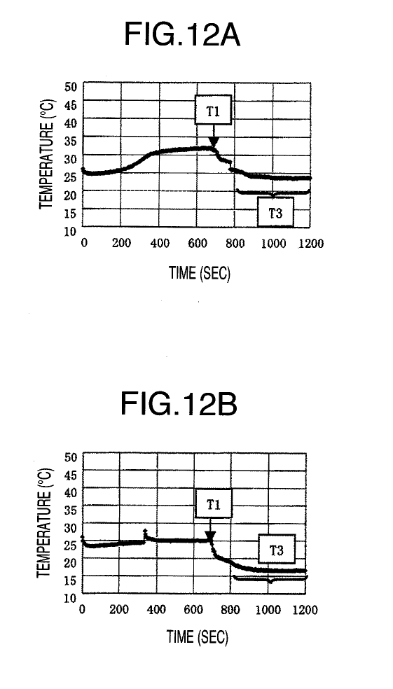

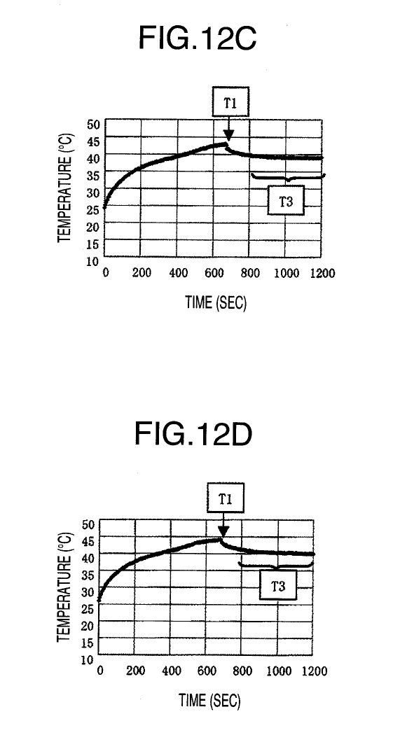

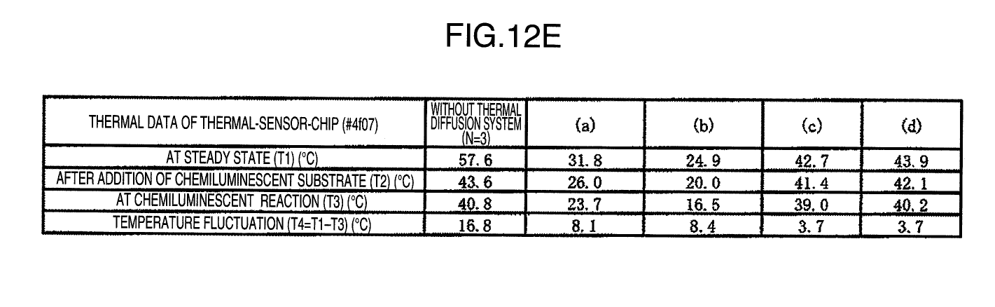

Next, the exothermal effect (temperature shift of sensor chip) of sensor chip heat by the thermal diffusion medium will be explained with reference to FIGS. 12A to 12E. FIGS. 12A to 12E represent the result of investigation of the temperature shift of the sensor chip in immunochromatography by the use of the sensor chip using the thermal diffusion system shown in FIGS. 11A and 11B. Horizontal axis represents the time and vertical axis does the temperature shift measured by using the sensor chip having a sensor mounted thereto. First, the exothermal effect of heat generated from the sensor chip is examined by using (a) a heat conduction sheet (material: silicone gel, thermal conductivity: 0.81 W/m·K) and (c) an α gel (material: a gel, thermal conductivity: 0.18 W/m·K) as the thermal diffusion medium. Next, to further improve the exothermal effect in (b) and (d), the exothermal effect is examined in the cases where a part of the thermal diffusion medium is brought into close contact with aluminum foil (material: aluminum, thermal conductivity: 236 W/m·K) in addition to (a) and (c). As shown in FIG. 12(e), in (a) and (c) where the heat conduction sheet is applied, the temperature is suppressed to 24.9 to 31.8° C. 12 minutes later (T1) at which the sensor-chip temperature gets stabilized. The temperature shift width (T4=T1−T3) resulting from the addition of the solution becomes small to 8.1 to 8.4° C. Similarly, in (c) and (d) where the αgel is applied, the temperature of T1 drops to 42.7 to 43.9° C. and the temperature of T4, to 3.7° C. The exothermal effect is confirmed in either case.

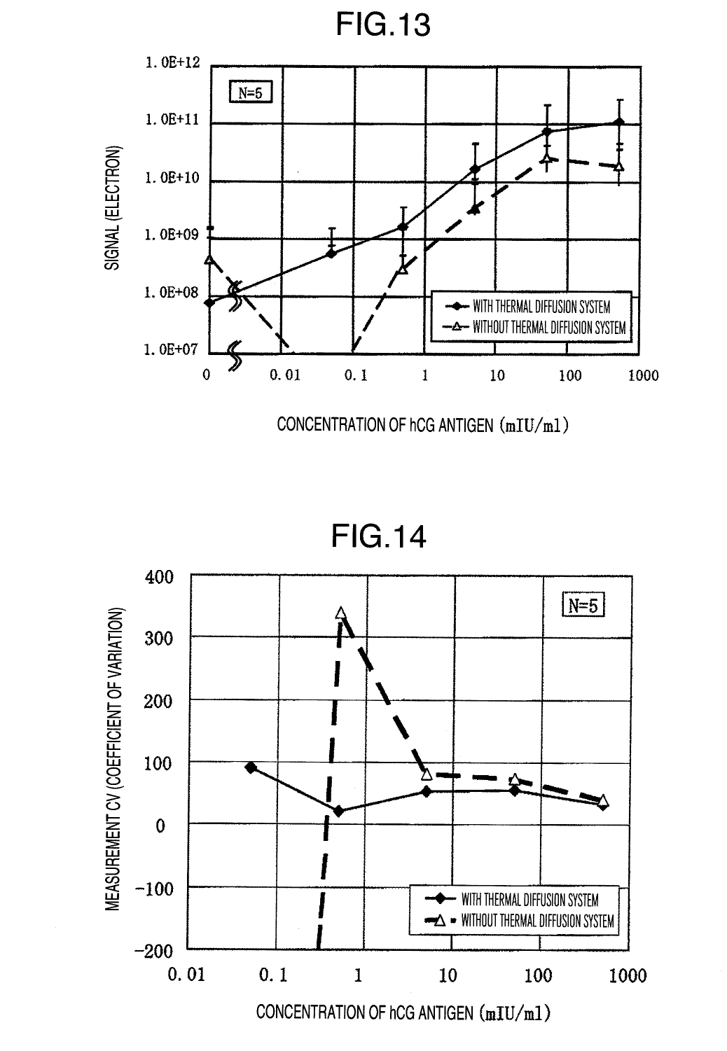

The effect of the thermal diffusion system shown in FIGS. 11A and 11B will be hereinafter explained with reference to FIGS. 13, 14, 15 and 16. First, the effect for the signal intensity will be explained with reference to FIG. 13. FIG. 13 shows a standard curve (N=5) of the hCG antigen prepared by immunochromatography applying a sensor chip. Horizontal axis represents an antigen concentration and vertical axis does a signal intensity (number of electrons). The term “signal intensity” determined hereby is the balance obtained by subtracting the luminescence intensity of the blank area from the luminescence intensity of the test area. When the exothermal measure of the sensor chip heat is taken by arranging the heat conduction sheet (with heat conduction sheet), the signal intensity at each antigen concentration rises and 0.5 mIUmI is detected as the detection lower limit in comparison with the case where the heat conductive sheet is not arranged (without heat conduction sheet).

The exothermal measure effect of the sensor chip with respect to measurement accuracy will be explained with reference to FIG. 14. FIG. 14 shows the graph prepared by determining the mean value of the signal intensity (MEAN), standard deviation (SD) and variance (CV) from the standard curve of the hCG antigen obtained in FIG. 13. When the heat conduction sheet is not arranged (without heat conduction sheet), variance (CV) at 0.5 to 5000 mIU5000 mIU/ml is as large as 40 to 100% but when the exothermal measure of the sensor chip by using the heat conduction sheet is executed, the variance (CV) can be reduced down to 30 to 50%.

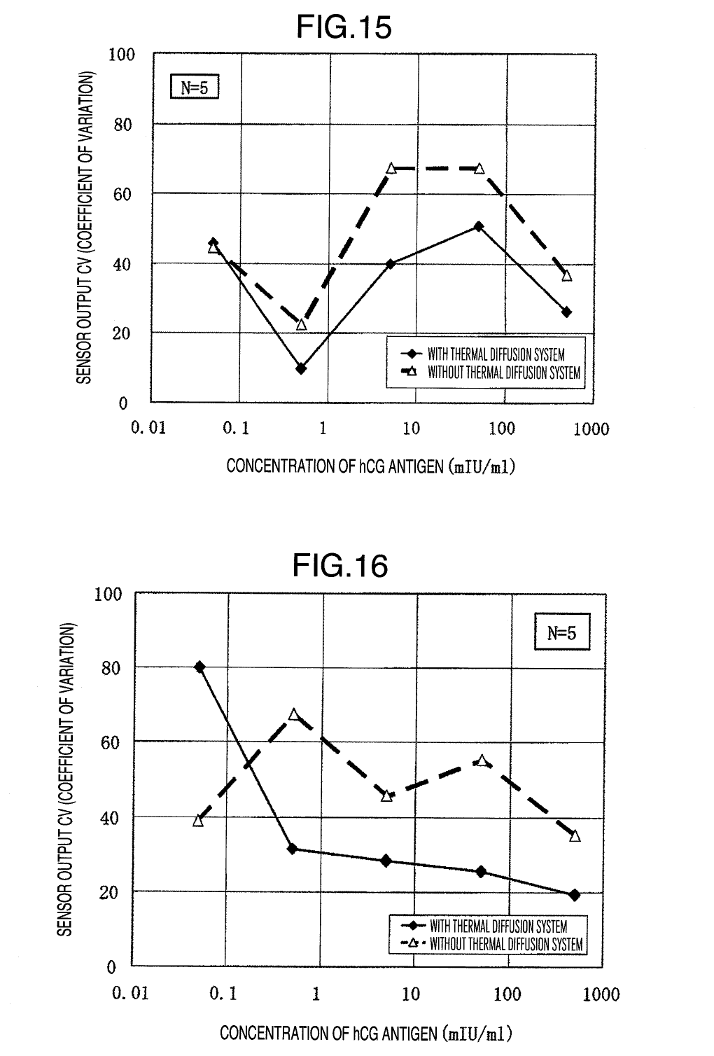

The exothermal measure effect of the sensor chip with respect to the sensor output variance in the test area and in the blank area will be explained with reference to FIGS. 15 and 16. The exothermal measure effect of the sensor chip with respect to measurement accuracy will be explained with reference to FIG. 14. FIG. 15 shows the graph prepared by determining the mean value of the luminescence intensity (MEAN), standard deviation (SD) and variance (CV) from the standard curve of the hCG antigen obtained in FIG. 13 and showing the comparison result of the sensor output variance in the test area. Similarly, FIG. 16 shows the comparison result of the sensor output variance in the blank area. The sensor output variance can be stabilized in both test area and blank area by arranging the heat conduction sheet. The term “sensor output” calculated hereby is the balance obtained by subtracting the sensor output in the blank area (photodiode dark current) from the sensor output in the test area. Therefore, measurement accuracy can be improved as the sensor output variance becomes smaller in each area. As for the increase of the signal intensity, it is believed that the improvement of the photodiode sensitivity, the drop of the proportion of inactivation of the antibody and the drop of the denaturation of the antigen in the immunoreactions or the decrease of the enzyme inactivation proportion in the enzyme reaction and the drop of the denaturation proportion of the base substrate material occurs.

It has been found out from the result shown in FIGS. 13, 14, 15 and 16 that the drop of measurement accuracy in immunochromatography using the sensor chip is associated with instability of the photodiode dark current that occurs owing to the temperature rise of the sensor chip. It has also been clarified that suppression of the temperature fluctuation of the sensor chip temperature by the exothermal diffusion system of the sensor chip heat by using the heat conduction sheet is effective as the counter-measure against this phenomenon.

### Embodiment 2

Integration Type of Sensor with Signal Processing Circuit:

FIG. 2A shows an embodiment of a sensing device applying a thermal diffusion system to a POCT device having integration type sensor. Main constituent elements of the thermal diffusion system are an integrated sensor chip 105, an external controller 209, a biological sample hold-back carrier 102, an antibody immobilized area 104 formed in the biological sample hold-back carrier 102 and a thermal diffusion medium 103. To make measurement, two kinds of antibodies (first antibody and second antibody) coupling peculiarly with a measurement object are employed. As a sample solution containing the measurement object is added drop-wise to the biological sample hold-back carrier 102, the measurement object is collected by the immobilized antibody (first antibody). When the second antibody modified by an enzyme catalyzing chemiluminescence is hereby added drop-wise, a sandwich structure composed of first antibody—measurement object—second antibody is formed and the enzyme is accumulated in the antibody immobilized area in accordance with the concentration of the measurement object. As chemiluminescence base material on which the enzyme exerts the catalytic action is caused to flow through the biological sample hold-back carrier 102, chemiluminescence occurs in the antibody immobilized area 104 and the sensor (photodiode in this embodiment) on the integration sensor chip 105 senses chemiluminescence corresponding to the measurement object. In this embodiment, the supply of electric power to the integration sensor chip 105 and the exchange of signals are made through wiring 106. FIG. 2B shows a functional block diagram of the sensing device. The integrated sensor 105 is controlled by a control command from a controller 209. The control command is demodulated by an interface block 213, is decoded by a control logical circuit block 212 and controls a sensor analog circuit 107 through a sensor interface (sensor IF). An antibody that peculiarly couples with the sensing object is immobilized in advance to the antibody immobilized area 104 as a part of the biological sample hold-back carrier 102. Signals of the sample and then the emission substrate detected on the biological sample hold-back carrier 102 are first amplified by the sensor analog circuit block 107 including an AD converter (ADC) and an amplifier and are then converted to digital electric signals. The digital sensing signal is encoded by the control logical circuit block 21, is modulated in the interface block 213 and is then transmitted to the external controller 209.

### Embodiment 3

Integrated Sensor Chip with Wireless Communication:

As an embodiment of a sensing system applying an exothermal structure to a POCT device having an integrated sensor, FIG. 3A shows a sensing system 121 applying the exothermal structure to a POCT device using wireless communication as means for supplying electromagnetic energy and transmitting/receiving the control command and measurement data. The system is constituted by an integrated sensor chip 108, a biological sample hold-back carrier 102, a reader coil 109, a reader 210 and a PC 217. Here, the sensor chip controlling function of the external controller 209 shown in FIGS. 2A and 2B is borne by the reader 210 and the PC 217. FIG. 3B shows a functional block diagram. The integrated sensor chip 108 is controlled by a control command from the PC 217 through the reader 210. The control command is demodulated by an RF circuit block 214 extending from a chip coil 216 connected to the sensor chip through a resonance circuit block 215, is decoded by a control logical circuit block 212 and controls a sensor analog circuit block 107 through a sensor interface (sensor IF). An antibody that peculiarly couples with the sensing object is immobilized in advance to an antibody immobilized area 104 as a part of the biological sample hold-back carrier 102. FIG. 3A depicts that the antibody is immobilized to only the antibody immobilized area 104 of the biological sample hold-back carrier 102. In practice, however, the antibody may be immobilized to both front and back surfaces or a part or the whole of a side surface besides the sensor area 202 of the sensor chip 108. A protective coat is formed on the surface of the sensing system shown in FIG. 3 with the exception of the sensor area 202. For, the antibody immobilized to the protective coat does not exert a significant influence on the operation of the sensing system. The detection signals of the sensor 202 of the sample on the biological sample hold-back carrier 102 and then the emission substrate are amplified by the sensor analog circuit block 107 including an AD converter (ADC) and an amplifier and are then converted to digital electric signals. The digital detection signal is encoded by the control logical circuit block 212, is demodulated in the interface block 213 and is transmitted to the external controller 209. The control logical circuit block 212 includes a UM circuit for storing or generating an identification number (UID: Unique Identifier) for specifying a specific sensor chip from among a plurality of sensor chips. Each sensor chip 108 has a different UID. Accordingly, a set of the PC 217 and the reader 210 can control a plurality of sensor chips 108. FIG. 3C shows a sensing system 122 for sensing a plurality of sensors by using one set of PC 217 and reader 210. The UID is transmitted to each sensor chip 108 from the PC 217 through the reader 210 while being carried by any of the electromagnetic wave, the change of a magnetic field and the change of an electric field. The UID reaches a plurality of sensor chips 108a and 108b placed inside the communication range of the coil and is received by an antenna 216 formed in each sensor chip 108. After rectification, the UID is collated with a unique UID written in advance to each center chip 108 through a demodulation circuit. Collation is made by a matcher inside the control logical circuit block 212 of each sensor chip 108. Power that is consumed by each circuit block in the sensor chip 108 and the sensor is supplied from a direct current source constituted by a rectification circuit, a smoothing circuit and a voltage regulator inside the RF circuit block 214 while any of the electromagnetic wave, the magnetic variation and the electric field change transmitted from the reader 210 is received by the chip coil 216 on the sensing system.

### Embodiment 4

Embodiment 4 will be explained with reference to FIGS. 11A and 11B. A test sensor having an optical sensor mounted thereto is arranged in such a manner as to keep close contact with an antigen 302 immobilized to an optical sensor area. A membrane component 301 as the biological sample hold-back carrier 301 serves as an area of the antigen-antibody reaction and the chemiluminescence reaction, and is preferably the environment that is hardly affected by the influences of heat generated from the sensor chip. Therefore, a heat conduction sheet 309 is brought into close contact with the back (opposite side of optical sensor area) 312 of the sensor chip as shown in FIG. 11A and a part of the heat conduction sheet 309 is brought into contact with slide glass 310 as the biological sample hold-back base material. Consequently, heat generated from the sensor chip can be emitted efficiently from the slide glass 310 through the heat conduction sheet 309 and thermal diffusion to the side of the biological sample hold-back carrier 301 can be suppressed.

The diffusion effect of heat generated from the sensor chips 308 and 3131 can be expected, too, by use of the construction in which the thermal diffusion medium 309 is brought into close contact with the biological sample hold-back carrier 301 in place of the sensor chips 303 and 313 and this construction is effective when temperature dependence of the reaction on the biological sample hold-back carrier 301 is relatively small.

### Embodiment 5

Embodiment 5 will be explained with reference to FIGS. 11A and 11B. In FIGS. 11A and 11B, a biological sample hold-back carrier 301 is arranged on slide glass 310 that serves as a supportive base, a wrap film 306 is arranged on the biological sample hold-back carrier 301 and sensor chips 308 and 313 are arranged on the wrap film 306. A heat conduction sheet 309 keeps close contact with the sensor chips 308 and 313 and with the slide glass 310 and thermal contact with the biological sample hold-back carrier 301. Thermal conductivity is 0.18 W/m·K for the biological sample hold-back carrier 301 (PES), 0.17 to 0.24 W/m·K for the wrap film 306 (main component: polypropylene/nylon), 168 W/m·K for the sensor chips 308 and 313 (main component: silicon), 1.0 W/m·K for the slide glass 310 (ordinary glass) and 0.8 W/m·K for the heat conduction sheet 309 (main component: silicone gel). Therefore, heat generated by the sensor chips 308 and 3131 is efficiently diffused from the slide glass 310 having high thermal conductivity through the heat conduction sheet 309. Here, the glass substrate is applied as the supportive substrate 310 but an exothermal effect similar to that of the heat conduction sheet 309 can be obtained by using the supportive substrate as the thermal diffusion medium as long as it has higher thermal conductivity than the heat conduction sheet 309.

### Embodiment 6

### Use of Plurality of Sensors:

Embodiment 6 will be explained with reference to FIGS. 11A and 11B. In FIGS. 11A and 11B, the measurement object sample (biological sample) and the reagents are added from a spacing between the test area (antibody immobilized area) 302 and the blank area 303. In FIG. 11A, the distance from an addition area 304 to each area (diffusion condition) is designed to be equal so that the chemiluminescence reactions in the test area 302 and the blank area 303 can have the same condition. Here, the design distance depends on the diffusion distance of the chemiluminescent substrate used (CDP-Star) in the membrane. In FIGS. 11A and 11B, the distance ranges (diffusion range) from the addition area 304 to the test area 302 and from the addition area 304 to the blank area 303 is within 0 to 5 mm under the condition where an enhancer (Nitro Block II, Tropix) that makes the surface of the porous structure of the membrane hydrophobic and promotes diffusion of the luminescence substrate (CDP-Atar) in the membrane. The position of the addition area for the measurement object sample (biological sample) and the reagents may well be the positions other than the spacing between the test area 302 and the blank area 303 as long as the distance condition to the test area 302 and the blank area 303 is the same.

### Embodiment 7

Integration with Thermal Diffusion Medium:

Embodiment 7 will be explained with reference to FIG. 7. A sensor chip 101 must keep thermal contact with a thermal diffusion medium 114 so as to diffuse exothermy of its own. The effect of the thermal diffusion medium 114 is greater when it keeps direct contact with an exothermal source than it keeps indirect contact and is greater when its contact area is greater. Therefore, the structure in which the sensor chip 101 is built in the thermal diffusion medium 114 as shown in FIG. 7 is a more effective structure.

### Embodiment 8

Integration with Biological Sample Hold-Back Carrier:

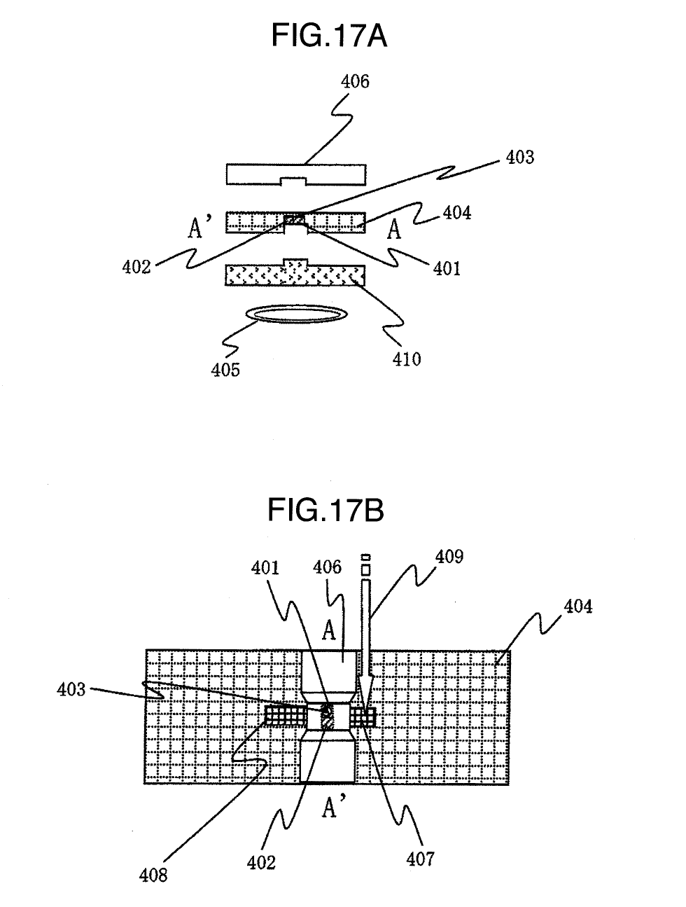

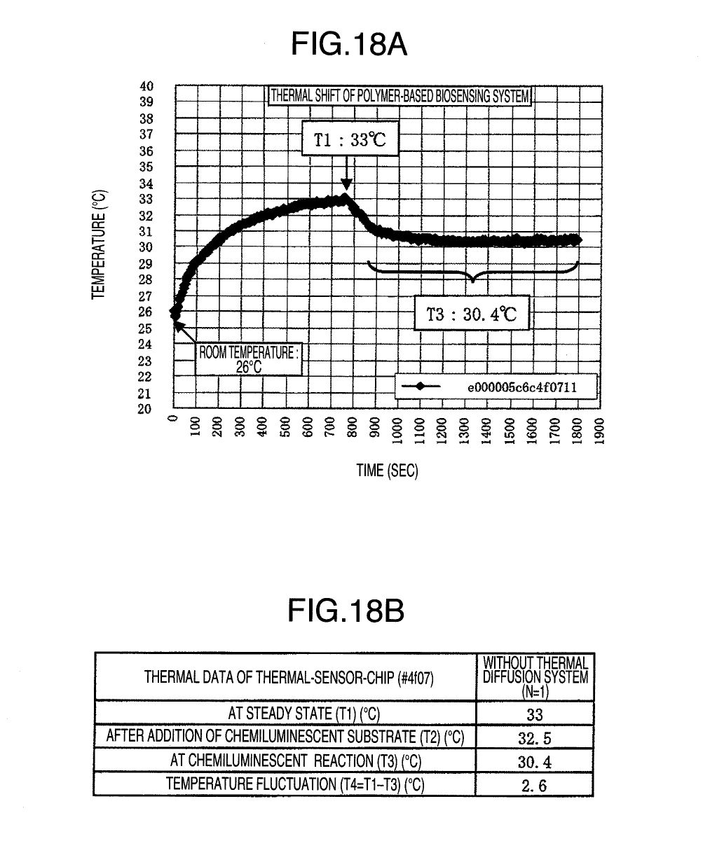

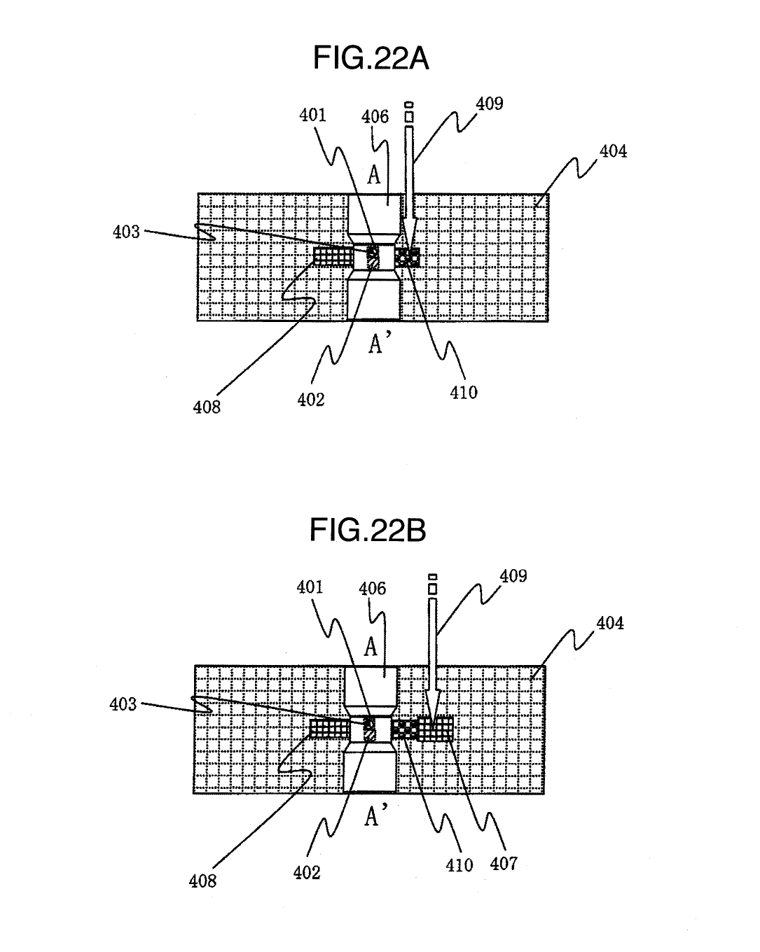

Embodiment 8 will be explained with reference to FIGS. 17A, 17B, 18A, 18B, 19A and 19B. FIGS. 17A and 17B show an immunochromatography sensing system by a sensor chip using a resin base material. FIG. 17A shows a sectional view that is viewed from a solution addition area 409 and FIG. 17B shows a plan view. Because the material of the resin base material 404 applied in FIGS. 17A and 17B is cyclic polyolefin (COC, thermal conductivity: 0.21 W/m·K), a structure in which a blank sensor chip 402 is buried into the resin base material 404 in the area where the test sensor chip 401 and the antibody are not immobilized immediately below the antibody immobilized area is applied. A flow pass 406 that keeps the biological sample and the reagent added from the solution addition area 409 and forms an efficient reaction field is formed on the resin base material 404. (A gap is secured in the drawing for ease of understanding). The flow pass 406 forms a pass having a height of 0.02 mm, a width of 5 mm and a length of 10 mm. The flow pass is formed on the test sensor chip 401 and the blank sensor chip 402 that are buried into the resin base material 404. A sample pad 407 is arranged on the side of a solution inlet 409 of the flow pass 406 and an absorbent pad 408 is arranged on the side of a solution outlet. A conjugate pad 410 containing an enzyme-labeled secondary antibody may be used as the sample pad 407 as shown in FIG. 22A. The conjugate pad 410 may be stacked as shown in FIG. 22B, too. The absorbent pad 408 disposed on the solution outlet side can obtain the function of controlling the flow velocity of the solution in addition to absorption of excessive biological sample and reagent that are added. A pump may be installed in place of the absorbent pad 408 to discharge the solution and to control the flow velocity. A sensor chip supportive base 410 is arranged below the resin base material 404 to bring the sensor chips 401 and 402 into close contact with the resin base material 404. The COC resin is applied to the sensor chip supportive base 410 in FIGS. 17A and 17B in the same way as the resin base material 404 but a more efficient exothermal effect can be obtained if a material has higher thermal conductivity. FIGS. 18A and 18B show the heat shift of the sensor chip itself in the sensing system applying the resin base material shown in FIGS. 17A and 17B by the use of the sensor chip to which the temperature sensor is mounted. When the supply of power is started from the reader coil 405 to the sensor chips 401 and 402, the chip temperature starts rising gradually from the room temperature 26° C. owing to exothermy of the sensor chips 401 and 402. The temperature (T1) at which the sensor chip temperature becomes constant and the photodiode dark current gets stabilized is 33° C. When the luminescence substrate solution is successively added, the temperature drops while depending on the solution temperature and the temperature (T3) during the luminescence reaction (mean of 1.00 to 1.800 seconds) drops to 30.4° C. The temperature rise of the sensor chips 401 and 402 from the room temperature is suppressed to 7° C. under the condition in which the resin base material 404 and the sensor chips 401 and 402 keep thermal contact in this way and the heat shift (T4=T1−T3) due to the addition of the solution is reduced to 2.6° C., too. The result given above represents that the resin base material 404 is the material having the thermal diffusion effect in the same way as the thermal diffusion medium.

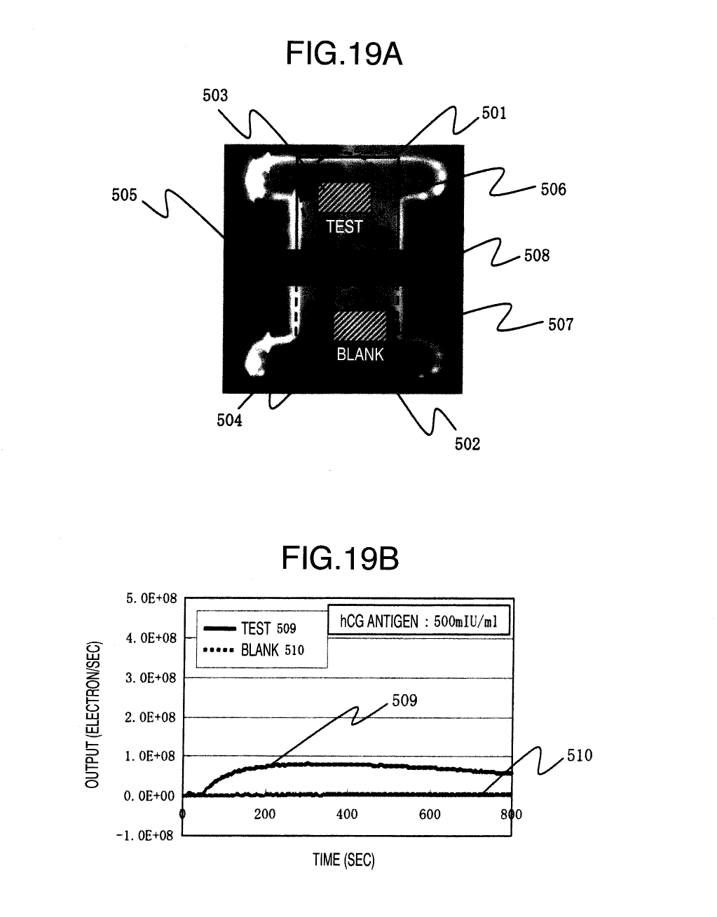

The resin is also the material that can be applied as the biological sample hold-back carrier when it is subjected to protein non-absorption treatment. FIG. 19 shows an example of sensor-chip immunochromatography using the COC resin base material subjected to the protein non-absorption treatment as the biological sample hold-back carrier. FIG. 19A shows the structure in which the back surface of the resin base material 505 is cut and the sensor chips 501 502 are buried. In the sensor chip 501, 502, the optical sensor area 503, 504 is buried towards the surface that is subjected to the protein non-absorption treatment. The area to which the antibody to the measurement object is immobilized is the test area 506 and the area to which the antibody to the measurement object is not immobilized is the blank area 507. FIG. 19B shows the result of immunoassay by using hCG antigen 500 mIU/ml as the measurement object. The horizontal axis represents the time and the vertical axis represents the photodiode output value transmitted from the photo sensor chip. Test 509 represents the result when the signal from the test area is sensed by the sensor chip 501 and blank 510 represents the result when the signal from the blank area 510 is sensed by the sensor chip 502. After luminescent substrate solution is added, a drastic output increase in test 509 and a slight output from blank 510 are detected and sensor chip immunoassay using the resin base material is verified.

It can be understood from the result given above that in the case of the material such as the resin base material having the similar effect as thermal diffusion medium, it can be integrated with the sensor chip as the biological sample hold-back carrier having the exothermal effect.

### Embodiment 9

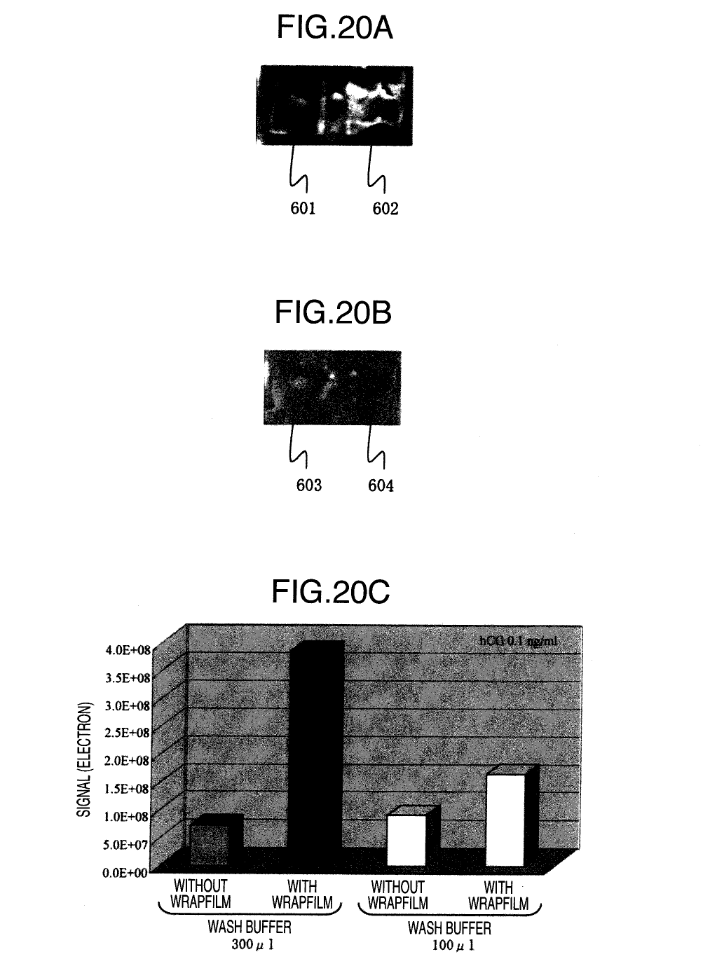

Embodiment 9 will be explained with reference to enhancement of diffusion of the chemiluminescent substrate solution and the improvement effect of uniformity by the wrap film shown in FIG. 20. FIG. 20 shows the data of comparison of the case where the wrap film 312 is arranged on the membrane component 307 as the biological sample hold-back carrier and the sensor chips 301 and 304 are arranged on the wrap film 312 and physically separated in the structure shown in FIG. 11 and the case where the sensor chips 301 and 304 are arranged directly on the membrane component 307 (without inserting the wrap film 312). In FIGS. 20A and 20B, sensor-chip immunochromatography is executed at hCG antigen concentration of 0.8 ng/ml to observe the distribution of the luminescent substrate on the membrane component through a CCD camera. In FIG. 20A where the wrap film is not arranged, the chemiluminescent substrate stays (no-flow) around the periphery of the sensor chip and an extremely high background occurs. In FIG. 20B where the wrap film is arranged on the membrane component, on the other hand, the no-flow phenomenon observed around the periphery of the sensor chip is eliminated. The data of sensor-chip immunochromatography at hCG antigen concentration of 0.1 ng/ml shown in FIG. 20C represents that the signal intensity increases owing to the disposition of the wrap film. From these results, the diffusion enhancement effect of the chemiluminescent substrate by the wrap film is confirmed.

Chemiluminescence assay by using the sensor chip having the mounted photo sensor has its feature in that the light source and the photo sensor are optically coupled with each other. Therefore, materials other than the wrap film may be used as well, so long as they are the materials, or have the structure, that does not impede optical coupling. Silicon photo diode (SPD) is applied to the photo sensor of the sensor chip of the invention and its sensitivity wavelength range (spectral response) is 190 to 1,100. Therefore, as long as the material does not impede the wavelength of this range, it does not affect the luminescent assay.

### Embodiment 10

Embodiment 10 will be explained with reference to FIGS. 12A, B, C, D and E. In FIGS. 12B and D, the exothermal effect when a part of the thermal diffusion medium is further brought into contact with aluminum having higher thermal conductivity is investigated. More concretely, FIG. 12B shows the result of investigation of the heat shift of the sensor chip when the heat conduction sheet is brought into close contact with slide glass and the terminal edge of the heat conduction sheet is further brought into close contact with the aluminum foil. FIG. 12D shows the result of investigation of the heat shift of the sensor chip when the heat conduction sheet is changed to the αgel and the heat shift is likewise investigated. In comparison with FIG. 12A where the heat conduction sheet is alone used, the temperature (T1) at which the temperature of the sensor chip become constant drops by 6.9° C. and the temperature shift width (T3) of the sensor chip drops by 0.3° C., too, in FIG. 12B where one of the ends of the heat conduction sheet is brought into close contact with the aluminum foil. It has been confirmed from the results given above that heat generated from the sensor chip can be diffused more efficiently when a part of the thermal diffusion medium is brought into contact with aluminum having high thermal conductivity. Though this embodiment uses aluminum, a higher exothermal effect can be expected by using materials (gold, silver, copper, iron, platinum, quartz, etc) having higher thermal conductivity than the thermal diffusion medium.

### Embodiment 11

As shown in FIG. 11, the thermal diffusion medium 309 is directed to diffuse heat so that exothermy from the sensor chips 308 and 313 is not transmitted as much as possible to the biological sample hold-back medium 301. Therefore, the material is preferably the one that has a greater thermal conductivity than the biological sample hold-back medium 301. In FIG. 11, a membrane component (PES membrane) is applied as the biological sample hold-back medium 301 and its thermal conductivity is 0.18 W/m·K. Therefore, the thermal diffusion medium 309 in the measurement using the sensor chips 308 and 312 preferably has thermal conductivity of 0.2 W/m·K or more.

### Embodiment 12

The signal intensity in immunochromatography applying the sensor chip is the balance obtained by subtracting the luminescence intensity (photodiode dark current) in the blank area from the luminescence intensity in the test area. Therefore, it is the premise that the measurement condition is the same between the test area and the blank area. The positions of the sensor chips 308 and 313 arranged in the test area 302 and the blank area 303 have the same distance from the inlet 304 as shown in FIG. 11. As to the heat conduction sheets 309 arranged on the sensor chips 308. 313, too, the heat conduction sheets having the same size (2.5 mm×25 mm) are arranged on the sensor chips 308 and 313. As described above, the exothermal structure using the heat conduction sheet 309 has the feature in that exothermal efficiency has the same condition between the test area 302 and the blank area 303.

### Embodiment 13

Embodiment 13 will be explained with reference to FIGS. 11A and 11B. Power supply and communication to the sensor chips 308 and 313 are made preferably in the form of inductive coupling of the reader coil 311 and the chip coil disposed below the biological sample hold-back carrier. In FIGS. 11A and 11B, the heat conduction sheet 309 using the silicone gel as the principal component is applied. Influences on power supply and communication to the sensor chips 308 and 313 in the system shown in FIGS. 11A and 11B are not observed as to the thermal diffusion medium consisting of the silicone gel as the principal component as shown in FIGS. 12A, B, C, D and E to FIG. 16.

### Embodiment 14

The sensor chip heat diffusion systems using the silicone gel and the αgel as the materials of the thermal diffusion medium have been explained in Embodiments 1, 10 and 13. Generally, materials having high conductance such as metals have high thermal conductivity but are not preferable because they impede transmission of carriers in wireless communication. Conductance of the silicone gel described above is about 5.0×10¹² Ω·cm and transmission of the carriers is not impeded. Generally, when the materials having conductance of not higher than 1.0×10⁶ Ω·cm are used as the thermal diffusion media, the heat diffusion effect can be kept while wireless communication is maintained.

### Embodiment 15

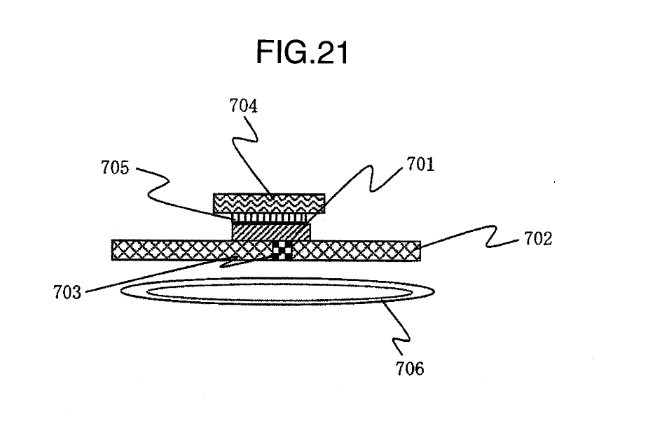

As an embodiment other than Embodiment 14, a high magnetic permeable material 705 may be used as shown in FIG. 21. Examples of the high magnetic permeable material include Fe—Ni alloy, ferrite, and so forth. According to this construction, lines of magnetic force can be allowed to penetrate through the coil of the sensor chip 701 even when a thermal diffusion medium 704 having high conductance is used.

### Embodiment 16

Embodiment 16 will be explained with reference to FIG. 13. FIG. 13 shows a standard curve of the hCG antigen prepared in accordance with Embodiment 1. The PES membrane applied as the biological sample hold-back carrier in FIG. 13 is furnished with surface modification suitable for the immobilization of proteins by covalent bonds. Similarly, the resin base material 404 shown in FIGS. 14A and 14B is subjected to protein non-adsorptive treatment, too, and becomes suitable as the biological sample hold-back carrier. FIGS. 18A and 18B show a measurement example of the temperature of the thermo sensor chip having a thermo sensor mounted thereto as an embodiment using the resin base material. Further, FIGS. 19A and 19B show the result of immunoassay using a photo sensor chip having a photo sensor mounted thereto. hCG antigen (concentration 500 mIU/ml) is used as the measurement object. In FIGS. 19A and 19B, a cyclic polyolefin (COC) resin 505 furnished with surface modification suitable for immobilization of proteins is applied as the biological sample hold-back carrier. An hCG-αsubunit antibody is immobilized and immuneassay is carried out by the sandwich method using alkaline phosphatase labeled hCG-βsubunit antibody in the same way as in Embodiment 1. Concavity is formed on the surface opposite to the modified surface of the resin base material and the sensor chips 501 and 502 are buried with their photo sensor areas 503 and 504 facing the modified surface. FIG. 19A shows a layout drawing of the resin base material of the test sensor chip 501 and the blank sensor chip 502. The antibody is immobilized in the test area 506 on the resin base material at the position that coincides with the photo sensor area of the sensor chip. To prevent cross-talk of luminescence occurring in the test area, an area 508 for absorbing or cutting off light is disposed between the test area 506 and the blank area 507. FIG. 19B shows the result of immunoassay at an hCG antigen concentration of 500 mIU/ml executed by using the construction shown in FIG. 19A. The horizontal axis represents the time and vertical axis does the photodiode output transmitted from the photo sensor chip. Test 509 shows the result of measurement of the signal from the test area made by the sensor chip 501 and represents that luminescence in the test area 506 can be detected.

Glass substrates, too, can be used as the biological sample hold-back carrier by applying the surface treatment. In substrates used for DNA chips, nucleic acid, etc, as the measurement object is immobilized on the surface modified glass substrate.

Sensor chips (semiconductors) can be used as the biological sample hold-back carrier by applying the surface modification suitable for the immobilization of protein or nucleic acids to the photo sensor area of the sensor chip.

### Embodiment 17

In FIGS. 12A, B, C, D, E and FIGS. 19A and 19B, immunochromatography is carried out by immobilizing the antibody protein to the biological sample hold-back carrier and using the antigen protein as the biological sample. Immunochromatography can be executed similarly by using blood or serum as the biological sample. Furthermore, screening of novel proteins and proteins having unknown functions can be made by using crude extracts of cells and tissues and fractionated proteins as the biological samples. When a target protein peculiar to a certain disease is used for the protein to be immobilized, screening of lead compounds as applicants of novels drugs can be made by using low molecular weight compounds for the biological sample.

When a nucleic acid (DNA, RNA, oligo) is immobilized as an example of immobilization of a biological sample other than the protein to the biological sample hold-back carrier, screening of a protein interacting peculiarly with a specific target sequence can be made, too.

### Embodiment 18

Embodiment 1 has explained that the diffusion operation of sensor chip heat by the heat conduction heat is effective for stabilizing the dark current of the photodiode of the sensor chip. FIGS. 12A, C and E represent the diffusion effect of the sensor chip by the thermal diffusion medium (heat conduction sheet, a gel). When no measure is taken for heat generated from the sensor chip as shown in FIG. 12E (without thermal diffusion system), temperature fluctuation (T4) of the sensor chip is 16.8° C. In immunochromatography of this sensor chip without thermal diffusion system, variance occurs in the dark current of the photodiode of the sensor chip as shown in FIG. 14. When heat of the sensor chip is diffused by bringing the heat conduction sheet into close contact with the sensor chip as shown in FIG. 12A, on the other hand, the temperature fluctuation (T4) of the sensor chip is decreased down to 8.1 to 8.4° C. and stabilization of the dark current can be observed in immunochromatography of the sensor chip employing this exothermal system. It can be concluded from the results described above that the temperature fluctuation (T4) before and after the reaction is preferably not higher than 10° C. in immunoassay using the sensor chip.

### Embodiment 19

As explained in Embodiment 8, the resin can be used as the biological sample hold-back carrier by applying the protein non-adsorptive treatment to the resin surface. Thermal conductivity of the COC resin used in FIGS. 17A, 17B and FIGS. 19A, 19B is 0.21 W/m·K and the resin base material has the effect similar to that of the thermal diffusion medium as shown in FIGS. 18A and 18B. Therefore, when the resin base material is applied, the biological sample hold-back carrier and the exothermal medium have the integrated system.

### INDUSTRIAL APPLICABILITY

The sensor chip according to the invention can be used for an extremely small and economical biosensing system integrated on a 2.5 mm×2.5 mm device on which a sensor, a signal processing circuit and a wireless communication circuit are integrated, for example. The invention can provide the sensor-chip immunoassay technology by combining this sensor chip with convenient immunochromatography technology. This technology is a novel inspection system that employs chemiluminescence for the detection of the antigen-antibody reaction, executes measurement by using the sensor chip having the mounted photo sensor and can make multi-item simultaneous measurement having high sensitivity and high quantitative analysis.

Although the invention has been described with reference to preferred embodiments thereof, it would be obvious to those skilled in the art that various changes and modifications can be made without departing from the spirit of the invention and within the scope of the appended claims.

### REFERENCE SIGNS LIST

101: sensor chip
102: biological sample hold-back carrier
102a: biological sample hold-back carrier a
102b: biological sample hold-back carrier b
103: thermal diffusion medium
103a: thermal diffusion medium a
103b: thermal diffusion medium b
104: antibody immobilized area
104a: thermal diffusion medium a
104b: thermal diffusion medium b
105: sensor chip with integrated signal circuit
106: wiring
107: control recorder
108: sensor chip with integrated signal circuit and wireless communication circuit
108a: sensor chip with integrated signal circuit and wireless communication circuit a
108b: sensor chip with integrated signal circuit and wireless communication circuit b
109: reader coil
110: thermal diffusion medium keeping contact with biological sample hold-back carrier
111: supportive base
112: sensor chip 2
113: thermal diffusion medium keeping uniform contact with plurality of sensor chips
114: thermal diffusion medium in the form of built-in sensor chip
115: heat diffusion type biological sample hold-back carrier
120: biosensing system containing sensor chip
121: biosensing system containing sensor chip
122: biosensing system containing sensor chip
210: sensor chip
202: sensor area
203: biological sample hold-back carrier
204: antibody immobilized area
205: thermal diffusion medium
206: absorbent pad
207: reader coil
208: inlet
209: external controller
210: reader
211: biological sample hold-back substrate
214: circuit block
215: resonance circuit block
216: chip coil
301: biological sample hold-back carrier
302: antibody immobilized area (test area)
303: blank area
304: solution inlet
305: absorbent pad
306: wrap film
307: photo sensor area
308: test sensor chip
309: thermal diffusion medium
310: supportive base
311: reader coil
312: back of sensor chip
401: test sensor chip
402: blank sensor chip
403: antibody immobilized area
404: resin base material
405: reader coil
406: flow pass
407: sample pad
408: absorbent pad
409: solution inlet
410: test sensor chip photo sensor area
402: blank sensor chip photo sensor area
501: test sensor chip
502: blank sensor chip
503: test sensor chip photo sensor
504: blank sensor chip photo sensor area
505: resin base material
506: antibody immobilized area (test area)
507: blank area
508: crosstalk prevention area
509: output value of sensor chip 501 (number of electrons/second)
510: output value of sensor chip 502 (number of electrons/second)
601: test area
602: blank area
603: test area
604: blank area
701: sensor chip
702: biological sample hold-back carrier
703: antibody immobilized area
704: thermal diffusion medium
705: high magnetic permeable material
706: reader coil

1. A sample assay chip comprising:
a hold-back carrier for holding an immobilized sample;
a sensor for detecting the reaction between a sample of a measurement object and said immobilized sample; and
a thermal diffusion medium for diffusing heat generated from said sensor;
wherein said thermal diffusion medium keeps thermal contact with said sensor.

2. A sample assay chip according to claim 1, wherein said sensor has a signal detection unit and a signal processing unit.

3. A sample assay chip according to claim 2, wherein said sensor further has a wireless communication unit.

4. A sample assay chip according to claim 1, wherein said thermal diffusion medium keeps thermal contact with said sample hold-back carrier or/and said sensor as said thermal diffusion medium keeps physical contact with them.

5. A sample assay chip according to claim 1, which further includes a base material for supporting said hold-back carrier, and wherein said thermal diffusion medium keeps contact with said base material.

6. A sample assay chip according to claim 1, which further includes a sensor for sample assay and a sensor for blank as said sensor, and has a groove for introducing a sample of said measuring object between said sensor for sample assay and said sensor for blank.

7. A sample assay chip according to claim 1, wherein said sensor is assembled in said thermal diffusion medium.

8. A sample assay chip according to claim 1, wherein said hold-back carrier is said thermal diffusion medium for diffusing heat generated from said sensor.

9. A sample assay chip according to claim 1, which further includes a layer for diffusing a sample of said measurement object added between said sensor and said hold-back carrier.

10. A sample assay chip according to claim 1, wherein said thermal diffusion medium includes an aluminum layer.

11. A sample assay chip according to claim 1, wherein said thermal diffusion medium has thermal conductivity of at least 0.2 W/m·K.

12. A sample assay chip according to claim 1, wherein said thermal diffusion medium equally diffuses heat generated from said sensor and heat generated from said sensor for blank.

13. A sample assay chip according to claim 1, wherein said thermal diffusion medium includes silicone gel.

14. A sample assay chip according to claim 1, wherein said thermal diffusion medium is formed of a material having electrical resistivity of at least 100 Ωcm.

15. A sample assay chip according to claim 1, wherein further includes a layer of high magnetic permeable materials between said sensor and said thermal diffusion medium.

16. A sample assay chip according to claim 1, wherein said hold-back carrier is formed of a material selected from the group consisting of a semiconductor, a porous membrane, glass and resin.

17. A sample assay chip according to claim 1, wherein said sample of said measurement object is either a biopolymer or a low molecular weight compound.

18. A sample assay chip according to claim 1, wherein a thermal shift of said hold-back carrier is not higher than 10° C. before and after said reaction.

19. A sample assay chip according to claim 1, wherein said hold-back carrier has thermal conductivity of at least 0.2 W/m·K.

20. A sensing system comprising:
a sample assay chip including a hold-back carrier for holding an immobilized sample, a sensor for detecting a reaction between a sample of a measurement object and said immobilized sample and a thermal diffusion medium for diffusing heat generated from said sensor, keeping thermal contact with said sensor; and
an external controller for exchanging signals with said sample assay chip.
